(19) United States
(12) Patent Application Publication
(10) Pub. No.: US 2006/0047245 A1
(43) Pub. Date: Mar. 2, 2006

(54) Catheter control unit

(76) Inventor: Ruchir Sehra, Del Mar, CA (US)

Correspondence Address: NEIL K. NYDEGGER;NYDEGGER & ASSOCIATES, 348 Olive Street, San Diego, CA, 92103, US

(21) Appl. No.: 10/924,450

(22) Filed: Aug. 24, 2004

(51) Int. Cl. A61M 31/00

(52) U.S. Cl. 604/95.04

(57) ABSTRACT

A catheter control unit includes a base member defining a common axis. A knob is mounted on one side of the base member, and a lever is mounted on the opposite side of the base member. A control cable is connected to the knob for proximal movement of the cable along the common axis in response to a rotation of the knob. Additionally, a pair of control wires is connected to the lever for opposite proximal and distal movements of the wires along the common axis in response to a rotation of the lever. The cable and wires are connected to a catheter to reconfigure the catheter as desired.

### FIELD OF THE INVENTION

The present invention pertains generally to hand-held control units. More particularly, the present invention pertains to extracorporeal control units that are capable of reconfiguring a catheter while the catheter is positioned in the vasculature of a patient. The present invention is particularly, but not exclusively, useful as a control unit for concertedly controlling the axial displacements of a control cable and a pair of control wires.

### BACKGROUND OF THE INVENTION

All interventional medical devices require some degree of directional control as they are being advanced into the vasculature of a patient. In particular, catheters typically require some contrivance for their steerability. For instance, various introducer sheath and guidewire systems have been proposed for this purpose. It also happens, however, that once a catheter has been advanced and properly positioned in the vasculature of a patient, it may still be desirable to somehow reconfigure the catheter. Although the inflation of a balloon on a balloon-catheter can be given as an example of a catheter reconfiguration, some surgical procedures may require a more contorted reconfiguration of the catheter tube itself. Regardless of the particular procedure, there is always a need for an effective control over the catheter that will cause it to be properly configured.

An immediately obvious purpose for extracorporeal catheter control is to provide steerability for the catheter as it is being advanced into the vasculature. Typically, this can be done simply by combining an in-plane bending of the catheter's distal end with an axial rotation of the catheter. Once the catheter is in situ, however, control over more than single-plane bending and axial rotation movements may be desirable. Indeed, some surgical procedures can benefit from an ability to simultaneously bend a catheter tube multi-directionally, in mutually perpendicular planes.

In light of the above, it is an object of the present invention to provide an extracorporeal control unit for multi-directionally bending a catheter tube while the tube is positioned in the vasculature of a patient. Another object of the present invention is to provide a control unit that can simultaneously bend a catheter tube in mutually perpendicular planes. Still another object of the present invention is to provide a hand-held control unit that is relatively simple to manufacture, is easy to use and is comparatively cost effective.

### SUMMARY OF THE INVENTION

In accordance with the present invention, a control unit for reconfiguring a catheter includes an elongated base member with a housing that defines a common axis. A knob is mounted to the housing on one side of the base member for selective rotation about a first control axis, and a lever is mounted to the housing on the opposite side of the base member for selective rotation about a second control axis. Preferably, the knob and the lever lie in respective planes that are mutually parallel to each other. Also, the first control axis of the knob is aligned to be substantially collinear with the second control axis of the lever. Further, both the first control axis and the second control axis are substantially perpendicular to the common axis.

For the present invention, a control cable and a pair of control wires are aligned together along the common axis. In more detail, the proximal end of the control cable is connected to the knob, and the proximal ends of each control wire are connected to the lever. This then leaves the respective distal ends of the cable and wires free for connection with an apparatus that can somehow be controlled by concerted axial movements of the cable and wires. As intended for one application of the present invention, these movements of the cable and wires are made to provide forces for reconfiguring a catheter, while the catheter is positioned in the vasculature of a patient.

Other aspects of the present invention include a handle that is formed on the base member to extend from the housing in a proximal direction along the common axis. Preferably, the handle has a width and is tapered with a decrease in the width in the distal direction. Further, the knob is preferably disc shaped and has a periphery that is formed with a plurality of indentations to facilitate its rotation. Still further, the lever is elongated and has a first tab and a second tab at its respective ends to facilitate rotation of the lever. For the preferred embodiment of the present invention, the handle, knob and lever are all made of plastic.

In the operation of the present invention, a rotation of the knob generates a pulling force on the control cable that will cause it to move along the common axis in a proximal direction. In a slightly different operation, the lever can be rotated in either a clockwise direction or in a counter-clockwise direction. Specifically, a rotation of the lever in either direction will cause a movement of one control wire in a proximal direction along the common axis, and a substantially simultaneous movement of the other control wire in a distal direction along the common axis.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a perspective view of a control unit in accordance with the present invention;

FIG. 2 is a top plan view of the control unit; and

FIG. 3 is a perspective schematic drawing of the structural configuration of control elements for the functional operation of the control unit.

### DESCRIPTION OF THE PREFERRED EMBODIMENTS

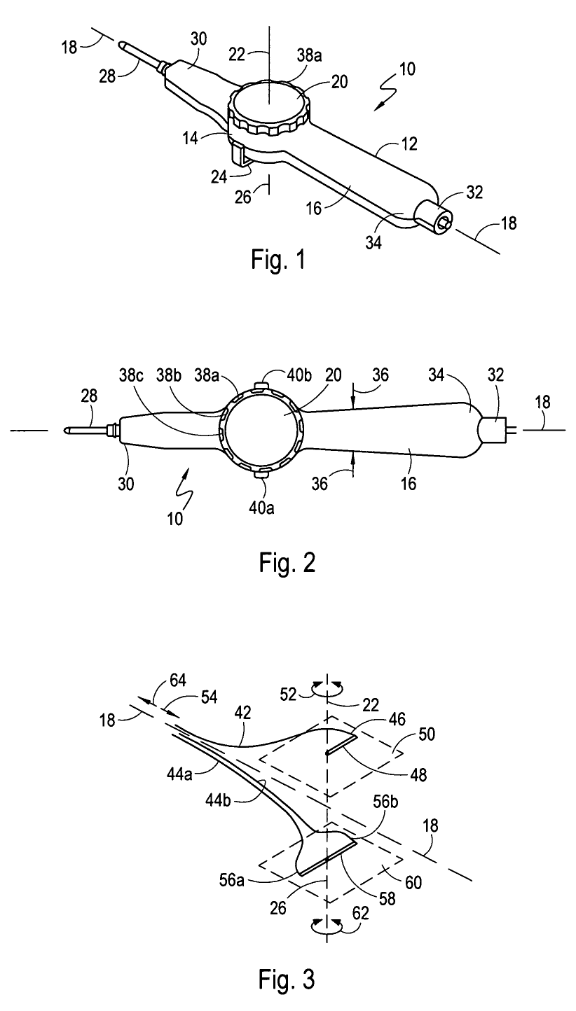

Referring initially to FIG. 1, a control unit in accordance with the present invention is shown and generally designated 10. As shown, the control unit 10 includes a base member 12 that is formed with a housing 14 and a handle 16. More particularly, as shown for the unit 10, the base member 12 (including housing 14 and handle 16) is elongated and defines a longitudinally oriented common axis 18. FIG. 1 also shows that the control unit 10 includes a knob 20 that is mounted on the housing 14 of base member 12 for rotation about a control axis 22. Further, it is shown that the control unit 10 includes a lever 24 that is mounted on the housing 14, opposite knob 20, for rotation about a control axis 26.

Still referring to FIG. 1, it will be seen that the control unit 10 may include a catheter tube 28 that is affixed to the distal end 30 of the base member 12. Further, the base member 12 may include a fixture 32 that is formed at its proximal end 34. As intended for the present invention, the catheter tube 28 is preferably flexible, and is capable of being multi-directionally bent in mutually perpendicular planes. As also intended for the present invention, the fixture 32 can be of any type known in the pertinent art that is capable of connecting the catheter tube 28 in fluid communication with a fluid source (not shown).

FIG. 2 shows that the handle 16 has a width 36, and that it is tapered with a decrease in the width 36 going in a direction from the proximal end 34 toward the distal end 30. FIG. 1 and FIG. 2 also show that the knob 20 has a plurality of indentations 38 that are formed at the periphery of the knob 20 for the purpose of facilitating the rotation of the knob 20 about the control axis 22. The indentations 38a, 38b and 38c shown in FIG. 2 are only exemplary. It can also be seen in FIG. 1 and FIG. 2 that the lever 24 is formed with a pair of opposed tabs 40a and 40b. More particularly, the tabs 40a and 40b are respectively located at the ends of the lever 24, and are oriented substantially parallel to the control axis 26. The purpose of the tabs 40a and 40b is to facilitate the rotation of lever 24 about the control axis 26.

In the operation of the present invention, the control unit 10 is used to control the configuration of the catheter tube 28. Specifically, this configuration control over the catheter tube 28 is accomplished by the concerted manipulation of a control cable 42, and a pair of control wires 44a and 44b (see FIG. 3). To do this, the control cable 42 and control wires 44a and 44b can be attached to the catheter tube 28 in any manner known in the pertinent art. Typically, it is expected that the control cable 42 and control wires 44a and 44b will somehow be positioned inside the lumen (not shown) of the catheter tube 28, and affixed to the catheter tube 28 at locations dictated by the proposed use of the catheter tube 28. The connections of the control cable 42 and control wires 44a and 44b to the control unit 10 are, however, more specific.

By cross-referencing FIG. 3 with FIG. 1 or FIG. 2, it will be appreciated that the proximal end 46 of the control cable 42 is attached to a connector 48 (exemplary only). In turn, the connector 48 is affixed to the knob 20. Accordingly, a rotation of the knob 20 will cause the connector 48 to rotate in a plane 50, about the control axis 22. The consequence of this is that a selective rotation of the knob 20 in the direction of arrow 52 about the control axis 22 will exert a tension force on the control cable 42 that will cause it to move in a proximal direction (indicated by arrow 54) along the common axis 18. A relaxation of the knob 20 can then be made to allow the control cable 42 to resume its unforced or neutral location along the common axis 18.

Still cross-referencing FIG. 3 with FIG. 1 or FIG. 2, it will also be appreciated that the respective proximal ends 56a and 56b of control wires 44a and 44b are attached to a connector 58 (exemplary only). The connector 58, in turn, is affixed to the lever 24. Accordingly, rotation of the lever 24 will cause the connector 58 to rotate in a plane 60 about the control axis 26. As indicated by arrow 62, the lever 24 and connector 58 can be rotated in either a clockwise or a counter-clockwise direction. In each case, whenever the lever 24 is rotated, the control wires 44a and 44b will be caused to move in opposite directions along the common axis 18. For example, consider a rotation of the lever 24 in a counter-clockwise direction (i.e. counter-clockwise as seen when looking down on plane 60 in FIG. 3). Such a rotation creates a tension in the control wire 44a that will move it in a proximal direction (indicated by arrow 54) along the common axis 18. At the same time, the control wire 44b will be moved in a distal direction (indicated by arrow 64) along the common axis 18. On the other hand, in response to a clockwise rotation of the lever 24, the control wire 44a will move in the distal direction (arrow 64) and the control wire 44b will move in the proximal direction (arrow 54).

When the control unit 10 of the present invention is used for the purpose of reconfiguring the catheter tube 28, it is intended that the knob 20 and lever 24 can be separately and independently operated. Nevertheless, the concerted operation of the knob 20 and lever 24 can be accomplished in several ways to create a variety of configurations for the catheter tube 28. In particular, the various contorted configurations created for the catheter tube 28 by the control unit 10 are envisioned to involve a multi-directional bending of the catheter tube 28 in mutually perpendicular planes.

While the particular Catheter Control Unit as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A control unit which comprises:
an elongated base member defining a common axis;
a knob mounted on said base member for selective rotation about a first control axis in a clockwise direction and in a counter-clockwise direction, wherein the first control axis is substantially perpendicular to the common axis of said base member;
a control cable aligned along the common axis and having a proximal end and a distal end, wherein the proximal end of said control cable is connected to said knob to cause a proximal movement of said control cable along the common axis in response to a rotation of said knob;
a lever mounted on said base member for selective rotation about a second control axis in a clockwise direction and in a counter-clockwise direction, wherein the second control axis is substantially perpendicular to the common axis of said base member; and
a pair of control wires aligned along the common axis, each having a proximal end and a distal end, wherein the proximal end of each control wire is connected to said lever to cause a proximal movement of one said control wire along the common axis in response to a rotation of said lever in the clockwise direction and to cause a proximal movement of the other said control wire along the common axis in response to a rotation of said lever in the counter-clockwise direction.

2. A control unit as recited in claim 1 wherein the first control axis and the second control axis are substantially collinear.

3. A control unit as recited in claim 1 wherein said base member is formed with a housing, said housing having a first side and a second side wherein said knob is mounted on the first side with the first control axis substantially perpendicular thereto, and wherein said lever is mounted on the second side with the second control axis substantially perpendicular thereto.

4. A control unit as recited in claim 3 wherein said base member is formed with a handle extending along the common axis in a proximal direction from said housing.

5. A control unit as recited in claim 4 wherein the handle has a width and is tapered with a decrease in the width in the distal direction toward said housing.

6. A control unit as recited in claim 1 wherein said knob is disc shaped and has a periphery wherein the periphery is formed with a plurality of indentations.

7. A control unit as recited in claim 1 wherein said lever is elongated and has a first end and a second end with the second control axis located substantially midway therebetween, and further wherein said lever has a first tab at the first end thereof and a second tab at the second end thereof, with the first tab and the second tab extending substantially parallel to the second control axis.

8. A control unit as recited in claim 1 further comprising a catheter tube, said catheter tube being attached to said base member and extending in a distal direction therefrom, with said control cable and said pair of control wires positioned therein.

9. A control unit as recited in claim 1 wherein said base member, said knob and said lever are made of plastic.

10. A hand-held device for selectively controlling concerted movements of a control cable and a pair of control wires along a common axis which comprises:
a base member;
a disc shaped knob mounted on said base member and defining a first plane, said knob being connected to the control cable and rotatable on said base member in the first plane about a first control axis to move the control cable along the common axis; and
a flat, elongated lever mounted on said base member and defining a second plane, said lever being connected to the pair of control wires and rotatable on said base member in the second plane about a second control axis to simultaneously move the control wires in opposite directions along the common axis, wherein the first plane and the second plane are substantially parallel to each other, and wherein the first control axis and the second control axis are substantially collinear with each other and are substantially perpendicular to the common axis.

11. A device as recited in claim 10 wherein said base member is formed with a handle extending along the common axis in a proximal direction from said housing.

12. A device as recited in claim 11 wherein the handle has a width and is tapered with a decrease in the width in the distal direction toward said housing.

13. A device as recited in claim 10 wherein said knob is disc shaped and has a periphery wherein the periphery is formed with a plurality of indentations.

14. A device as recited in claim 10 further comprising a catheter tube, said catheter tube being attached to said base member and extending in a distal direction therefrom, with said control cable and said pair of control wires positioned therein.

15. A device as recited in claim 10 wherein said base member, said knob and said lever are made of plastic.

16. A method for selectively controlling concerted movements of a control cable and a pair of control wires in a proximal direction and in a distal direction along a common axis which comprises the steps of:
providing a base member, with a disc shaped knob mounted thereon to define a first plane, the knob being connected to the control cable and rotatable on the base member in the first plane about a first control axis, and with a flat, elongated lever mounted thereon to define a second plane, the lever being connected to the pair of control wires and rotatable on the base member in the second plane about a second control axis, wherein the first plane and the second plane are substantially parallel to each other, and wherein the first control axis and the second control axis are substantially collinear with each other and are substantially perpendicular to the common axis;
rotating the knob to move the control cable, wherein the control cable is aligned along the common axis and has a proximal end and a distal end with the proximal end thereof connected to the knob to cause a movement of the control cable in the proximal direction along the common axis in response to a rotation of the knob;
alternately rotating the lever in a clockwise direction and in a counter-clockwise direction, wherein the pair of control wires are aligned along the common axis with each having a proximal end and a distal end, with the respective proximal end thereof connected to the lever to simultaneously move the control wires in opposite directions along the common axis in response to a rotation of the lever.

17. A method as recited in claim 16 further comprising the step of selectively moving the base member in the proximal direction and in the distal direction relative to the common axis.

18. A method as recited in claim 16 further comprising the step of turning the base member in rotation about the common axis.

19. A method as recited in claim 16 wherein the base member is formed with a handle extending along the common axis in a proximal direction, wherein the handle has a width and is tapered with a decrease in the width in the distal direction, wherein the knob is disc shaped and has a periphery formed with a plurality of indentations, and further wherein the lever is elongated and has a first end and a second end with the second control axis located substantially midway therebetween, and the lever further has a first tab at the first end thereof and a second tab at the second end thereof, with the first tab and the second tab extending substantially parallel to the second control axis.

20. A method as recited in claim 16 wherein the base member, the knob, and the lever are made of plastic.
